(19) United States
(12) Patent Application Publication
(10) Pub. No.: US 2002/0098598 A1
(43) Pub. Date: Jul. 25, 2002

(54) Method for tracking compounds in solution phase combinatorial chemistry

(76) Inventors: David L. Coffen, San Diego, CA (US); Xiao-Yi Xiao, San Diego, CA (US); Yi Hu, San Diego, CA (US)

Correspondence Address: Colleen J. McKiernan, Brown Martin Haller & McClain, 1660 Union Street, San Diego, CA, 92101, US

(21) Appl. No.: 10/056,257

(22) Filed: Jan. 24, 2002

Related U.S. Application Data
(60) Provisional application No. 60/263,789, filed on Jan. 24, 2001.

(51) Int. Cl. G06F 19/00; G01N 33/536

(52) U.S. Cl. 436/536; 702/19

(57) ABSTRACT

A method for the generation of chemical libraries using machine-readable passive tagging systems is described. The preferred embodiment uses radiofrequency tags or one- or two-dimensional bar codes to track vials and the contents therein throughout the course of the synthesis. This abrogates the need for specialized combinatorial chemistry equipment, allowing the use of standard laboratory baths, ovens, shakers and racks, as well as manual fluid handling techniques.

### CROSS-REFERENCES TO RELATED APPLICATIONS

This application claims the benefit of priority of U.S. provisional application Serial No. 60/263,789 filed Jan. 24, 2001 which is incorporated herein by reference in its entirety.

### FIELD OF THE INVENTION

The invention relates to a method for tracking synthesis of individual compounds during solution phase combinatorial chemical library production using a machine readable passive tagging system.

### BACKGROUND OF THE INVENTION

Advances in the field of molecular biology have led to the identification and characterization of a number of disease-related biomolecular targets from various biological systems. The identification of such targets has allowed for the development of high throughput screens for biologically active compounds that can act as agonists or antagonists of receptors and inhibitors of enzymes. Extracts from plants and other organisms often contain biologically active compounds; however, the purification of small molecules from natural sources is a slow and sometimes arduous process. Similarly synthesis of compounds individually for use in high throughput screens is also impractical.

A promising approach to the synthesis of large collections of diverse molecules is combinatorial chemistry. Using such methods large libraries of molecules having different chemical compositions can be synthesized en masse. Combinatorial methods entail a series of steps in which sets of chemical reagents are sequentially reacted with sets of starting compounds. The complexity of the library is given by the arithmetic product of the number of reagents chosen for each step of the synthesis and can therefore be quite large.

A central issue of combinatorial chemistry is preserving the identity of each member of a compound library throughout the multistep process required to create a library. A typical process entails the use of several building block (reagent) sets and usually two or more synthetic organic reactions. In addition, various unit operations may be necessary to add specific reagents and catalysts, effect dissolution of reactants, provide for heating, cooling, and agitation, quench reactive reagents or intermediates, filter out insoluble byproducts, separate inorganic salts from desired organic products, purify products, and transfer them into formats suitable for chemical analysis and biological testing. After testing, active compounds must be identified for larger scale synthesis and further testing. Thus it is critical in the preparation of a compound library that a reliable method for maintaining the identity of each library member be used at all stages.

Initial methods for identification of active compounds from combinatorial libraries involved the isolation, purification and analysis of the compound using the process of deconvolution. This was both tedious and time consuming. Subsequently, methods were developed to track compounds as they were synthesized, by addition of tags to beads in solid phase systems or the use of grids for solution phase synthesis.

For the preparation of compound libraries using solid phase organic synthesis (SPOS), several highly reliable methods for synthesis and tracking have been developed. These methods depend on the fact that in SPOS, the product molecules are covalently bound to insoluble resin beads at every step except the final step when the library members are cleaved from the solid support. The commonly used methods include labeling porous containers used to encapsulate aliquots of beads with one- or two-dimensional bar codes (U.S. Pat. Nos. 4,631,211 and 6,136,274), inserting a radiofrequency transponder capable of generating a unique signal in a porous container (e.g. U.S. Pat. No. 6,087,186), and labeling beads by co-synthesis of chemical tags (e.g. U.S. Pat. No. 6,001,579; Ohlmeyer et al., 1993. Proc. Natl. Acad. Sci. 90:10922-6). However, co-synthesis of tags can limit available chemistry options, as the synthesis of the compound of interest cannot interfere with the synthesis or cause the degradation of the tag, or vice versa. The use of machine readable tags and labels has proven to be particularly valuable for SPOS-based combinatorial chemistry because they have made it possible to develop electronically controlled automation devices for sorting and manipulating individual library members at numerous stages, making it possible, for example, to capture the productivity enhancement of the split and pool technique in the production of single compound libraries.

In sharp contrast to SPOS, solution phase combinatorial library production depends on the “spatial address” of each sample at every stage to preserve sample identity (e.g. U.S. Pat. Nos. 5,736,412 and 5,712,171). This is typically achieved by arraying samples in a two dimensional grid, often with the 8×12 pattern of a microtiter plate as the basic unit of the grid. While microtiter plates can be used directly for library production with some types of organic reactions, requirements of scale, heat transfer, resistance of the plate to organic solvents, etc., have driven the widespread development of reaction block technology to meet these requirements (e.g. U.S. Pat. No. 5,609,826). Such a reaction block uses replaceable reaction vials supported in the block which has fittings that facilitate robotic manipulation. Identification of the compounds relies on the location of the compound in the spatial array. Vials must remain in their designated locations in the array to preserve the identity of their contents. Additionally the system requires automated fluid handling devices and other specialized equipment. As many of the solution phase synthesis methods are designed to fit into a 96-well format to be compatible with robotic fluid handling devices, the number of compounds that can be synthesized is relatively low as compared to other methods.

### SUMMARY OF THE INVENTION

The invention is a method for solution phase combinatorial chemistry which utilizes the techniques of machine readable passive tagging including, but not limited to, radiofrequency transponders and two-dimensional bar codes. During synthesis, vials may be sorted and arranged by any method without losing the identity of the vial. Tracking of the synthesis of compounds is no longer dependent on spatial restrictions or on the presence of chemical tags that need to be modified to track the steps of synthesis.

The advantages of the new method are that fixed spatial arrays are no longer required during library production. The sample size of individual library members is no longer bounded by the size of vials or tubes that fit into blocks or racks for automated processing. Standard laboratory ovens, baths and shakers can be used to provide heating, cooling, or agitation. All vials that require a particular building block or reagent can be grouped by hand or automated sorting methods, thereby simplifying reagent additions to the point where manual transfer with multichannel pipettors is at least as efficient as the use of programable fluid handling devices. Parallel and serial unit operations can be used interchangeably within a library production protocol; e.g. the vials can be loaded in racks for parallel centrifugal evaporation, or processed serially for liquid-liquid extraction in automated equipment. Deletion of library members for quality control or other reasons at any stage before the final format no longer entails reformatting consequences. Limitations on potential reagents and reaction conditions are minimal as synthesis or maintenance of tags is not required. Possible reagents include essentially all reagents currently in use for organic synthesis that can be reacted by the methods currently in use.

### BRIEF DESCRIPTION OF THE DRAWINGS

The present invention will be better understood from the following detailed description of an exemplary embodiment of the invention, taken in conjunction with the accompanying drawings in which like reference numerals refer to like parts and in which:

FIG. 1. Schematic of solution phase library synthesis using directed sorting.

FIG. 2. An example of chemical reactions of a single member of a library in a combinatorial synthesis.

FIG. 3. Cyclic anyhydrides used in synthesis of a combinatorial library.

FIG. 4. Secondary amines used in synthesis of a combinatorial library.

FIG. 5. Aryl amines used in synthesis of a combinatorial library.

FIG. 6. A generalized reaction showing the synthesis of the members of the spiro oxindole library.

### DETAILED DESCRIPTION AND PREFERRED EMBODIMENTS

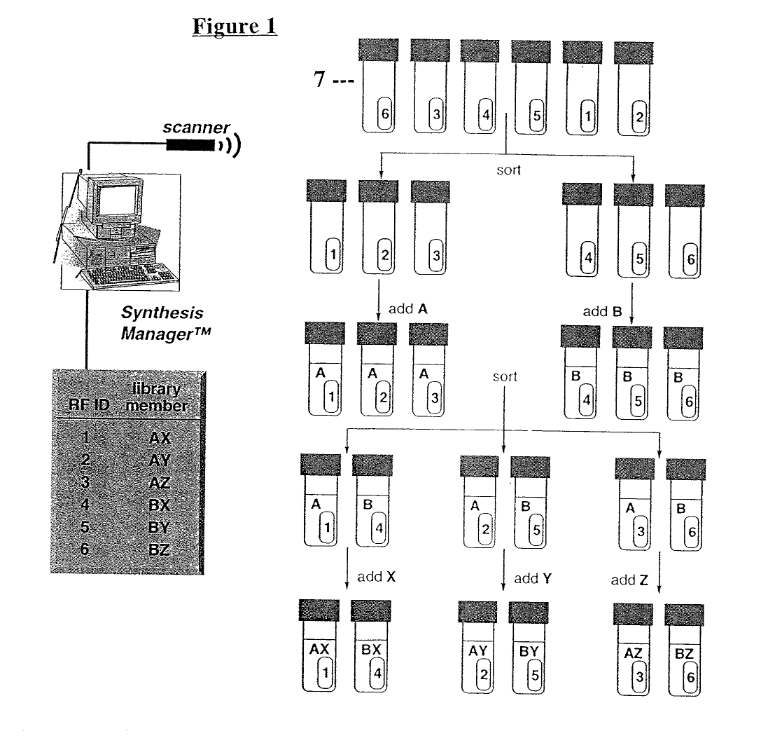

The method of the invention is simplistically diagramed in FIG. 1 and detailed in Example 1, which illustrate the sorting and resorting steps that are guided by the use of machine readable tags. The method of the invention is exemplified by the description of the synthesis of two combinatorial libraries by the method of the invention.

### EXAMPLE 1

In FIG. 1, vials (7) are labeled with tags (numbered 1 though 6) and sorted into two groups containing vials with labels 1-3 and 4-6. Vials in one group are charged with reactant A (vial numbers 1-3) and vials in the other group are charged with reactant B (vial numbers 4-6), each reactant in its appropriate solvent. The vials are then sorted into three groups, each pair containing a vial each with reactant A and reactant B. A second compound is added to each of the vials each in its appropriate solvent per the synthesis plan. Reactant X is added to vials numbered 1 and 4. Reactant Y is added to vials numbered 2 and 5. Reactant Z is added to vials numbered 3 and 6. Vials are subjected to the reaction conditions depending on the reactants. Upon completion of the synthesis, all six possible combinations of the two sets of reactants exist, AX, AY, AZ, BX, BY, BZ.

Products are identified by a unique tag on the vial which is assigned to a specific product at the beginning of synthesis such that the vial is directed through a series of steps according to the chemical synthesis plan (e.g. 1=AX, 2=AY, etc). Upon completion of synthesis, aliquots are transferred to multiwell daughter plates for further analysis (e.g. structure, yield, purity).

### EXAMPLE 2

Bis-amide Library Production.

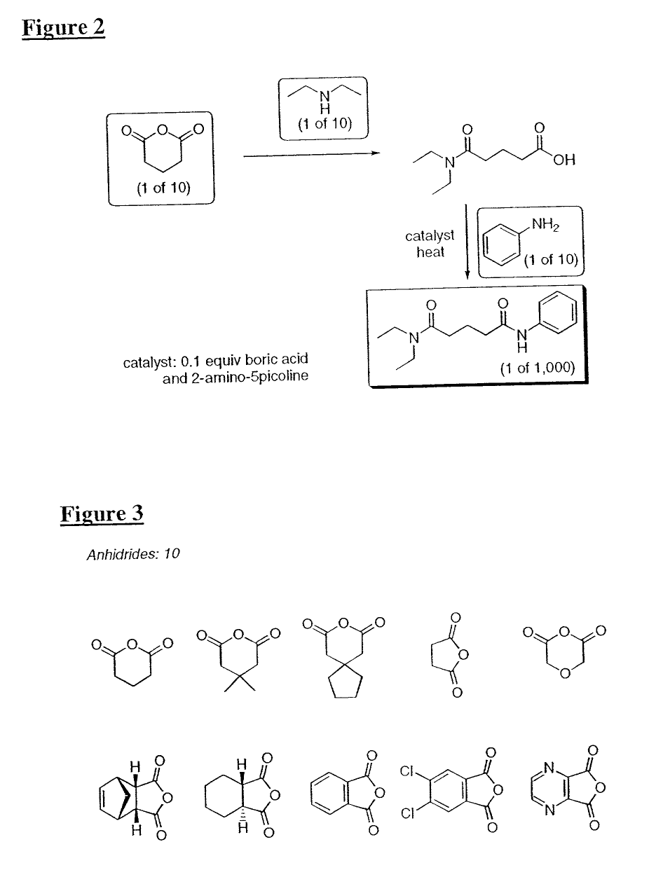

A library of bis-amides was generated by the reaction exemplified in FIG. 2. A cyclic anhydride was reacted with a secondary amine to produce a carboxyamide intermediate. Solvent was evaporated and an aryl amine was added in the presence of 10 mole percent boric acid and 2-amino-5-picoline to generate the final product. Specifically, one thousand 8 ml Teflon-lined screw-cap glass vials were each charged with a glass coated IRORI radiofrequency tag, each bearing a unique Rf code. Using IRORI operating software and an AccuTag sorter, each vial was assigned to a particular product and all vials were sorted into ten groups containing 100 in each group. The vials in each group were then charged with 1 ml of a 0.125M stock solution of one of the ten cyclic anhydrides shown in FIG. 3 in tetrahydrofuran. The anhydride corresponding to each group is set by the computer generated synthesis plan.

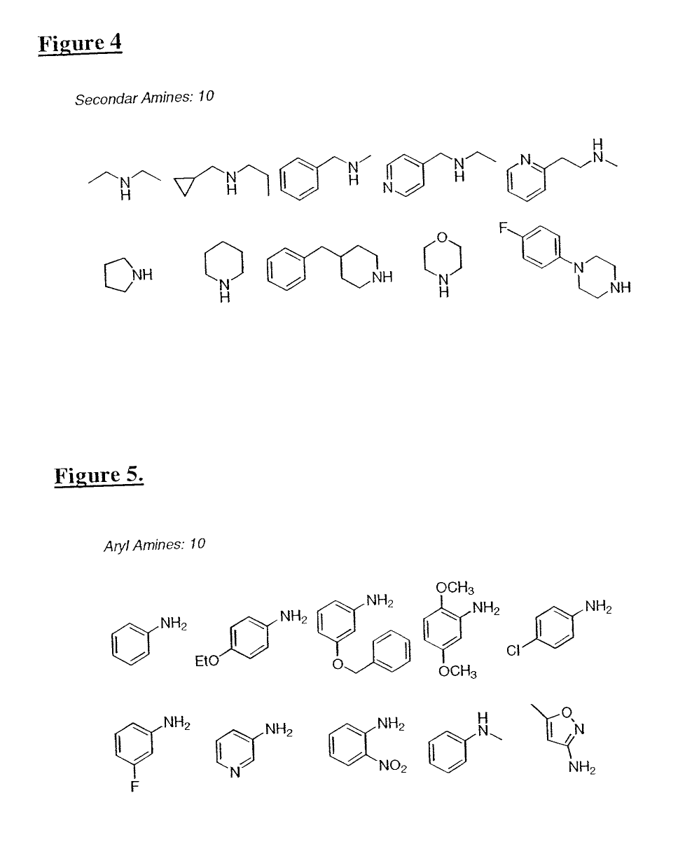

The groups of vials were then pooled and resorted into ten new groups of 100 in which each group comprised ten vials containing each of the ten cyclic anhydrides. All the vials in each group were then charged with 1 ml of a 0.125 M methylene chloride stock solution of one of the ten secondary amines shown in FIG. 4, following the synthesis plan.

The vials were shaken at 25° C. for 14 hours to generate the carboxyamide intermediates. Alternatively the vials were stirred using the Rf tags as stir bars. The caps were removed and the solvents evaporated in vaccuo.

The groups of vials were then pooled and resorted into ten groups of 100 in which each group contained one vial containing each of the 100 carboxyamide intermediates. All the vials in each group were then charged with 1 ml of 0.125M toluene solution of one of the ten aryl amines shown in FIG. 5. Each vial was also charged with 10 μl of a 1.25M solution of boric acid in N,N-dimethylformamide and 1.0 ml of a 0.0125M solution of 2-amino-5-picoline in toluene. The capped vials contained in a wire basket were shaken at 25° C. for 12 hours then heated in a standard laboratory oven at 110° C. for 14 hours.

The vials were allowed to cool and then sorted into 42 sets of 24 vials, with 16 vials in the last set. Each set of 24 was placed in a 24-position rack with the location of each vial correlated to a structure in an SD file and a microtiter plate location. The caps were removed and the solvents evaporated in a centrifugal evaporator. The products were taken up in methanol and transferred to microtiter plates (master plates) using a Gilson fluid handler. A set of daughter plates was prepared for liquid chromatography and mass spectrometry analysis of the libraries.

The radiofrequency tags were recovered from the vials for reuse in another library.

### EXAMPLE 3

Spiro Oxindole Library Production.

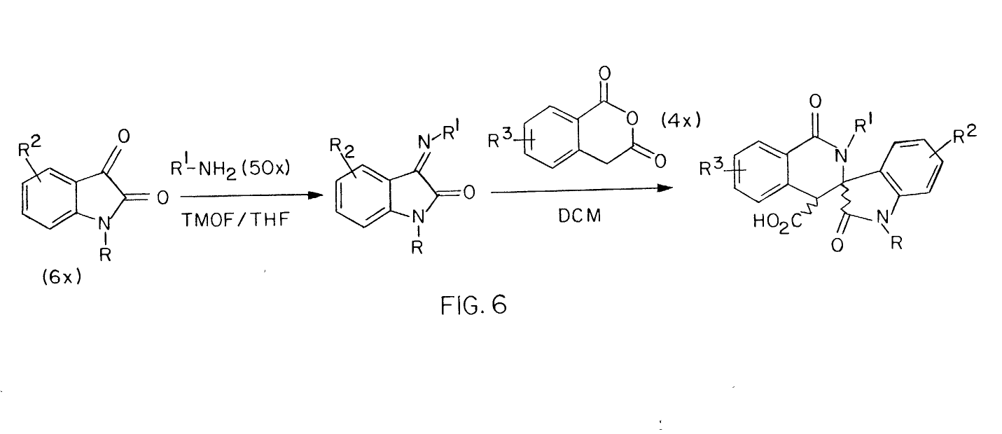

A library of spiro oxindoles was generated using the solution phase combinatorial chemistry method detailed above and shown in the reaction in FIG. 6. Fifteen primary amines were reacted with eight istatin derivatives, solvents were removed and the products were reacted with eight homophthalic anhydride solutions to generate the spiro oxindoles.

Primary amines were dissolved in dry 5-hydroxymethylene tetrahydrofolate (THF) at a concentration of 0.5M. Amines with lower solubility in THF were dissolved at concentrations as low as 0.2M. If such a dilution was still insufficient to dissolve all materials, water was added up to 10% to allow for complete solution. Istatin derivatives were dissolved in 0.1-0.2M THF depending on their specific solubility characteristics. Homophthalic anhydrides were prepared prior to use by refluxing the corresponding diacids derivatives in dichloromethane (DCM). As this is not typically sufficient to dissolve the anhydrides, a suspension was prepared by sonication that can be pipetted manually (concentration 0.1M).

For synthesis of a 960 compound library-8ml Teflon-lined screw cap glass vials were labeled with a unique, scanner readable two-dimensional bar code. Vials were assigned to a specific product that maps to a specific series of reagents and reaction steps according to the synthesis plan. Vials were sorted into 15 groups of 64 vials and were each charged with 0.1 mmole of each of the 15 primary amines. The vials were resorted into eight new groups of 120 each containing eight vials of each of the primary amines according to the synthesis plan. All of the vials in each group were charged with 0.1 mmole of each of the istatin derivatives. Finally, 1.5 ml of trimethylorthoformate (TMOF) was added to each of the 1000 vials.

Vials were gently shaken overnight at room temperature. Caps were removed and solvents were evaporated in vaccuo.

Reaction residues were suspended in 1.0 ml of 50% THF/DCM. Vials were then pooled and resorted into eight new groups of 120 in which each group comprised one vial containing each of the 120 different imine intermediates. All of the vials in each group were charged with 0.1 mmole of one of the eight homophthalic anhydrides according to the synthesis plan.

Vials were gently shaken overnight at room temperature. Caps were removed and solvent was evaporated in vaccuo.

Compounds were dissolved or suspended in 1.5 ml methanol and aliquots were manually transferred to 96-well plates for analysis.

The reagents used in the synthesis were:

Amines:

1. 3-Methoxypropylamine

2. 2-(2-methoxyphenyl)ethylamine

3. 2-Cyclopropylethyl amine

4. Cyclopropylamine

5. 2-(3-Chlorophenyl)ethylamine

6. Cyclohexylamine

7. 4-Phenylbutylamine

8. 2-(3-Pyridinyl)ethylamine

9. 4-(1-benzyl)piperidinylamine

10. Ethylamine

11. 2-Phenoxyethylamin

12. 2-(4-Fluorophenyl)ethylamine

13. Cyclobutylamine

14. N-(3-Aminopropyl)carbamic acid tert-butyl ester

15. Isopropylamine

Isatines:

1. Isatin

2. 5-Chloroisatin

3. 5-bromoisatin

4. 5-Fluoroisatin

5. 5-methylisatin

6. 5-Trifluoromethoxyisatin

7. 1-methylisatin

8. 5-Nitroisatin

Homophthalic anhydrides

1. Homophthalic anhydride

2. 6-Fluorohomophthalic anhydride

3. 6-Methoxyhomophthalic anhydride

4. 6-Chlorohomophthalic anhydride

5. 7-Methylhomophthalic anhydride

6. 7-Fluorohomophthalic anhydride

7. 7-Methoxyhomophthalic anhydride

8. 7-Chlorohomophthalic anhydride

Although an exemplary embodiment of the invention has been described above by way of example only, it will be understood by those skilled in the field that modifications may be made to the disclosed embodiment without departing from the scope of the invention, which is defined by the appended claims.

We claim:

1. A method for producing combinatorial chemical libraries using solution phase combinatorial chemistry comprising labeling vials with unique, machine readable tags, performing at least two chemical reactions and tracking the reactions using the machine readable tags.

2. A method as in claim 1, wherein the machine readable tags comprise radiofrequency (Rf) encoded transponders.

3. A method as in claim 1, wherein the machine readable tags comprise one- and two-dimensional bar codes.

4. A method as in claim 1, wherein the reactions comprise a chemical synthesis plan.

5. A method as in claim 1, wherein machine readable tags encode information links which relate to each individual container and its contents to a specific compound in the chemical synthesis plan.

6. A method as in claim 1, wherein machine readable tags guide machines and automation devices employed for tracking, sorting and manipulating individual containers throughout the synthesis plan.
